(19) United States
(12) Patent Application Publication
(10) Pub. No.: US 2025/0176856 A1
(43) Pub. Date: Jun. 5, 2025

(54) STROKE DETECTION

(71) Applicant: Covidien LP, Mansfield, MA (US)

(72) Inventors: Ajay S. Kadrenahally, Rancho Santa Margarita, CA (US); Ravi Sadasivuni, Aliso Viejo, CA (US); Adam G. Hendry, San Clemente, CA (US); Erik P. Fahimi, Aliso Viejo, CA (US); Shivram Kashyap Sridhar, Minneapolis, MN (US)

(21) Appl. No.: 18/962,485

(22) Filed: Nov. 27, 2024

Related U.S. Application Data
(60) Provisional application No. 63/605,249, filed on Dec. 1, 2023.

(51) Int. Cl. A61B 5/08; A61B 5/00; A61B 5/05

(52) U.S. Cl. CPC A61B 5/0826; A61B 5/05; A61B 5/0816; A61B 5/7282; A61B 5/74

(57) ABSTRACT

In some examples, an example system includes a contactless sensor and processing circuitry. The contactless sensor is configured to generate a sensor signal indicative of a breathing pattern of a patient. The processing circuitry is configured to receive the sensor signal; detect, based on the sensor signal, a stroke event; and generate, in response to detecting the stroke event, a notification indicative of the stroke event.

### CROSS-REFERENCE TO RELATED APPLICATIONS

The present application claims the benefit of U.S. Provisional Application Ser. No. 63/605,249, filed Dec. 1, 2023, which is incorporated herein by reference in entirety.

### TECHNICAL FIELD

The present disclosure generally relates to strokes, and to stroke detection.

### BACKGROUND

Stroke is a medical condition that can cause permanent neurological damage, complications, and death. Stroke may be characterized as the rapidly developing loss of brain functions due to a disturbance in the blood vessels supplying blood to the brain. The loss of brain functions can be a result of ischemia (lack of blood supply) caused by thrombosis or embolism. During a stroke, the blood supply to an area of a brain may be decreased, which can lead to dysfunction of the brain tissue in that area.

Detecting and treating strokes as soon as possible promotes the effectiveness of stroke therapy received by patients. A variety of approaches exist for treating patients undergoing a stroke. For example, a clinician may administer anticoagulants or may undertake intravascular interventions such as thrombectomy procedures to treat ischemic stroke. However, such treatments may be frequently underutilized and/or relatively ineffective due to the failure to timely identify whether a patient is undergoing or has recently undergone a stroke.

### SUMMARY

In general, this disclosure describes devices, systems, and techniques for detecting a stroke event based on a breathing pattern of a patient determined via a contactless sensor (e.g., Doppler ultrasound). The stroke event can be, for example, a prediction of stroke, an onset of stroke, an ongoing stroke, a ceased stroke, or a patient condition indicative of a physiological condition associated with stroke. Ischemia in cranial blood vessels that can cause a stroke may also cause the patient to exhibit an abnormal breathing pattern indicative of a stroke event. The contactless sensor is configured to generate a signal indicative of a breathing pattern of a patient, e.g., when the patient is asleep, and processing circuitry is configured to receive the signal and determine whether the signal indicates a breathing pattern indicative of a stroke event. In some examples, the processing circuitry generates a notification in response to detecting the stroke event. The notification can be, for example, an audible, visual, or tactile notification, a signal sent to another party, such as a clinician, emergency service provider, or patient caretaker, or the like.

Visible signs of strokes may include facial drooping, weakness of limbs, or speech impediment. However, if a patient is asleep, such signs may be not readily apparent or communicated. Devices, systems, and techniques according to the present disclosure facilitate detection of stroke based on changes in breathing patterns, which may reduce or prevent the need to rely on audio or visual indicia of stroke. For example, stroke may be detected without monitoring facial features or speech. Further, contactless sensors may be used to sense the breathing patterns, which may facilitate contactless or remote monitoring of stroke. A notification indicative of stroke may be transmitted to a patient caretaker, a clinician, an emergency service provider, or a first responder, which may promote a relatively rapid response and treatment of stroke.

In some examples, an example system includes a contactless sensor and processing circuitry. The contactless sensor is configured to generate a sensor signal indicative of a breathing pattern of a patient. The processing circuitry is configured to receive the sensor signal; detect, based on the sensor signal, a stroke event; and generate, in response to detecting the stroke event, a notification indicative of the stroke event.

In some examples, an example technique includes receiving, by processing circuitry, a sensor signal indicative of a breathing pattern of a patient from a contactless sensor and detecting, by the processing circuitry, based on the sensor signal, a stroke event. The technique may further include generating, by the processing circuitry, in response to detecting the stroke event, a notification indicative of the stroke event.

In some examples, an example non-transitory computer readable storage medium includes program instructions configured to cause processing circuitry to receive a sensor signal indicative of a breathing pattern of a patient from a contactless sensor. The program instructions are further configured to cause the processing circuitry to detect, based on the sensor signal, a stroke event. The program instructions are further configured to cause the processing circuitry to generate, in response to detecting the stroke event, a notification indicative of the stroke event.

In some examples, an example sleep monitoring device includes a Doppler sensor, processing circuitry, and a housing securing the Doppler sensor and the processing circuitry. The Doppler sensor may be configured to generate a sensor signal indicative of a breathing pattern of a patient with or without physically contacting the patient. For example, the Doppler sensor may be spaced from the patient, or may contact skin of the patient, or may contact an intermediate article in contact with or worn by the patient. The processing circuitry is configured to receive the sensor signal; detect, based on the sensor signal, a stroke event; and generate, in response to detecting the stroke event, a notification indicative of the stroke event.

The details of one or more examples of the techniques of this disclosure are set forth in the accompanying drawings and the description below. Other features, objects, and advantages of the techniques will be apparent from the description and drawings, and from the claims.

### BRIEF DESCRIPTION OF DRAWINGS

FIG. 1 is a block diagram illustrating an example system including a contactless sensor and a computing device configured to detect a stroke event.

FIG. 2 is a block diagram illustrating an example configuration of the contactless sensor of FIG. 1.

FIG. 3 is a block diagram illustrating an example configuration of the computing device of FIG. 1.

FIG. 4 is a block diagram illustrating an example configuration of a computing system that operates in accordance with one or more techniques of the present disclosure.

FIG. 5 is a flow diagram illustrating an example technique for detecting a stroke event.

FIG. 6 is a flow diagram illustrating an example technique for detecting a stroke event by at least determining an irregular breathing pattern.

### DETAILED DESCRIPTION

The present disclosure describes systems and techniques for detecting a stroke event, which may be or include a prediction of stroke, an onset of stroke, an ongoing stroke, a ceased stroke, or a patient condition indicative of a physiological condition associated with stroke. Patients may exhibit irregular breathing patterns in response to different disorders or conditions. For example, a patient may exhibit centralized periodic breathing (CPB) after bilateral strokes associated with disturbed consciousness. CPB may include cyclic fluctuations in breathing drive, hyperpneas alternating with apneas (absence of breathing) or hypopneas in a gradual waxing-and-waning fashion. CPB may include other irregular breathing patterns such as Cheyne-Stokes pattern. For example, Cheyne-Stokes pattern during sleep, associated with unilateral stroke or acute stroke, includes crescendo-decrescendo respirations followed by a period of apnea.

Other irregular breathing patterns may include Kussmaul, Biot's, or ataxic patterns. The Kussmaul pattern, associated with diabetic ketoacidosis, includes a deep and sighing respiratory pattern. The Biot pattern is associated with damage to the pons of the brainstem due to stroke or trauma, and includes deep respirations interrupted by apnea. Ataxic patterns include a high irregularity in breathing, including irregular pauses and increasing periods of apnea.

In some examples, an example system includes a contactless sensor and processing circuitry. The contactless sensor is configured to generate a sensor signal indicative of a breathing pattern of a patient, e.g., without physically contacting the patient. For example, the contactless sensor may be a Doppler sensor. The processing circuitry may be configured to receive the sensor signal; detect, based on the sensor signal, a stroke event; and generate, in response to detecting the stroke event, a notification indicative of the stroke event. For example, the processing circuitry may analyze the sensor signal and detect the stroke event based on the analysis. In some examples, the processing circuitry may determine a patient characteristic based on the sensor signal (for example, a breathing pattern such as CPB or another irregular breathing pattern), and detect the stroke event based on the patient characteristic.

In some examples, a system includes a sleep monitoring device including a Doppler system for detecting CPB or other irregular sleep patterns indicative of strokes or other neurological disorders. The system is configured to detect irregular sleeping patterns and transmit a notification, e.g., to a connected device, such as a smart phone, a smart watch, or the like. The system or the connected device may be configured to automatically send a notification or initiate communication with a clinician, a family member, a friend, a patient caretaker, or an emergency service provider. In some examples, the sleep monitoring device is a stand-alone connected device that itself sends the notification to or initiates communication indicating that the patient is suffering from an episode of stroke. The sleep monitoring device may be configured to be placed on, secured to, or held by one or more of a stand, an article of furniture, or any suitable support. For example, the sleep monitoring device may be configured to be placed on furniture (e.g., a table, a dresser, or a shelf) or secured to a portion of a bed (e.g., to a headrest or a siderail). In some examples, a wearable device may include the sleep monitoring device or the Doppler system.

In some examples, the system may be configured to detect a breathing pattern associated with a non-stroke event or condition, for example, sleep apnea. Thus, the system may distinguish irregular breathing patterns indicative of stroke from irregular breathing patterns indicative of other conditions. In some examples, the system may generate a notification indicative of detection of (or lack of detection) of stroke in addition to other conditions, such as sleep apnea.

Using a contactless sensor may reduce intrusion into the patient's sleep, for example, by reducing or avoiding contact with the patient. The contactless sensor may also continue to perform in response to patient movement that may tend to remove or displace alternative sensors or devices that may be attached to or in constant contact with the patient. Further, a contactless sensor may be powered or charged without intruding into the patient's sleeping space, for example, by spacing wiring or accessories needed for powering or charging from the patient. Moreover, the contactless sensor may be replaced with another type of sensor or another unit of the sensor (for example, for cleaning, maintenance, or clinical reasons) without touching the patient or interrupting the patient's sleep. The contactless sensor may also be able to monitor a relatively larger area of the patient's body by being spaced from the body, for example, in contrast with a device or a sensor attached to a particular location on the patient's body.

In some examples, a wearable, implantable, or contact sensor configured to contact the patient may be used instead of, or in addition to, the contactless sensor. For example, the wearable, implantable, or contact sensor may be configured to generate the sensor signal indicative of one or more of the breathing pattern or a physiological parameter of the patient. In some examples, the wearable, implantable, or contact sensor includes a wearable device, a external or implantable medical device (e.g., an electrical stimulation device, a pacemaker or a cardiac monitor). In some examples, the pacemaker or the cardiac monitor includes a Doppler system.

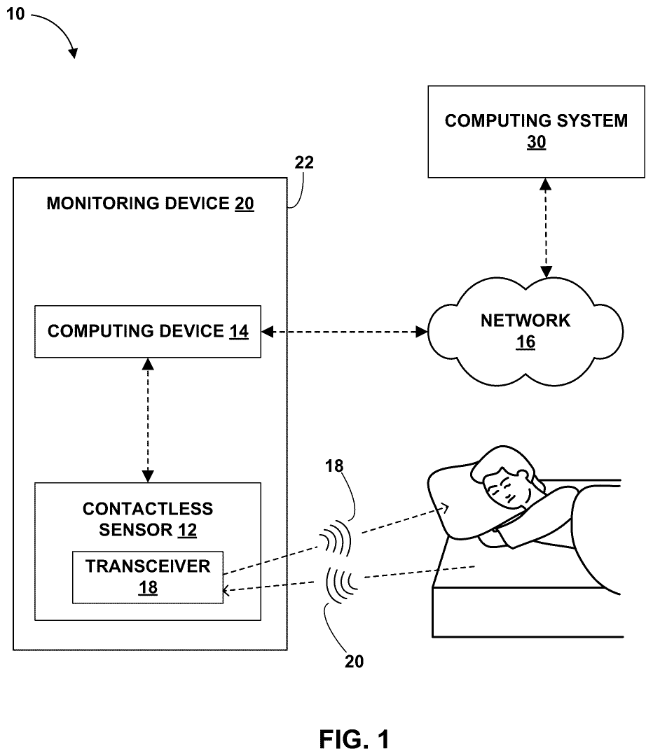

FIG. 1 is a block diagram illustrating an example system 10 including a contactless sensor 12 and a computing device 14 configured to detect a stroke event. Contactless sensor 12 is configured to generate a sensor signal indicative of a breathing pattern of a patient. Computing device 14 may include any suitable device, such as, but not limited to, a smart watch, a smart phone, a tablet, a laptop computer, a desktop computer, a smart speaker, or a sleep monitor.

Computing device 14 is configured to receive the sensor signal. For example, contactless sensor 12 may transmit the sensor signal to computing device 14 (e.g., via a wired or wireless communication channel), or to another computing system which in turn may transmit the sensor signal to computing device 14. Thus, computing device 14 may receive the sensor signal as a local signal from contactless sensor 14, or a remote signal from another computing device or computing system that is indicative of the sensor signal. Computing device 14 is further configured to detect, based on the sensor signal, a stroke event. For example, computing device 14 may extract one or more components, such as a frequency, a wavelength, an amplitude, or a Doppler shift, from the sensor signal.

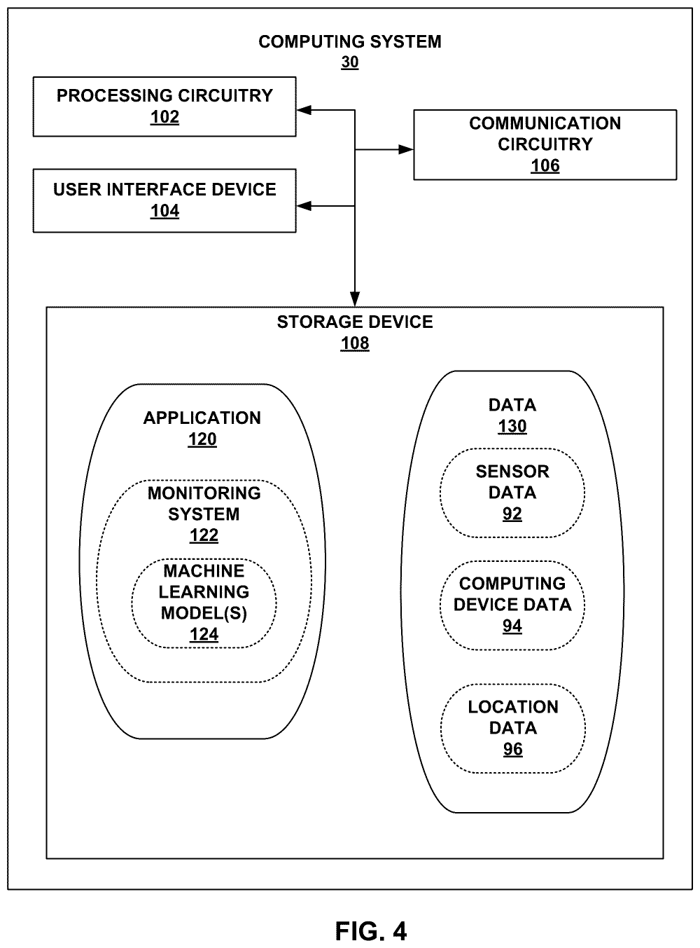

Computing device 14 is further configured to generate, in response to detecting the stroke event, a notification indicative of the stroke event. In some examples, the notification can be an audible, visual, or tactile notification, a signal sent to another party, such as a clinician, emergency service provider, or patient caretaker, or the like. In some examples, computing device 14 is configured to generate the notification by at least transmitting the notification via a network 16, for example, a wired network or a wireless network. In some examples, computing device 14 is configured to transmit the notification to a computing system 30 (described with reference to FIG. 4), for example, via network 16.

Network 16 may include one or more computing devices (not shown) and circuitry, such as one or more non-edge switches, routers, controllers, gateways, security devices such as firewalls, intrusion detection, and/or intrusion prevention devices, servers, cellular base stations and nodes, wireless access points, bridges, cable modems, application accelerators, or other network devices. Network 16 may include one or more networks administered by service providers, and may thus form part of a large-scale public network infrastructure, e.g., the Internet. Network 16 may provide computing devices, such as computing device 14 or computing system 30, access to the Internet, and may provide a communication framework that allows the computing devices to communicate with one another.

In some examples, network 16 includes a private network that provides a communication framework that allows computing device 14 to communicate with computing system 30 and/or other systems or devices, but isolates one or more of these devices or data flows between these devices from devices external to the private network for security purposes. In some examples, the communications between computing device 14 and other devices, such as devices of computing system 30, are encrypted.

In some examples, computing device 14 is further configured to determine, based on the sensor signal, the breathing pattern. For example, the patient may exhibit a regular or irregular breathing pattern. The irregular breathing pattern may include Cheyne-Stokes breathing, ataxic breathing, Kussmaul breathing, Biot's breathing, or some other breathing pattern, and computing device 14 may determine the particular breathing pattern exhibited by the patient. For example, computing device 14 may match the breathing pattern with a plurality of template breathing patterns, by comparing signal characteristics (for example, frequency, amplitude, or peak width, inter-peak intervals) with predetermined thresholds. Further, computing device 14 may detect, based on the breathing pattern, the stroke event. For example, computing device 14 may be configured to determine, based on the breathing pattern, an irregular breathing event. The irregular breathing event may be indicative of an abnormal patient condition and/or an occurrence of a medical event that may require medical attention. In some examples, the irregular breathing event may be indicative of occurrence of a stroke event. For example, the irregular breathing event may include a centralized periodic breathing event, which may be indicative of the stroke event. In some examples, the breathing pattern is indicative of a Cheyne-Stokes breathing event, which may in turn be indicative of the stroke event. Thus, computing device 14 may be configured to determine, based on the irregular breathing event, the stroke event.

The stroke event may include a prediction of stroke, an onset of stroke, an ongoing stroke, a ceased stroke, or a patient condition indicative of a physiological condition associated with stroke. Thus, computing device 14 may be able to generate notification indicating that stroke is possible, probable, or imminent, or that stroke has commenced or is ongoing, or that stroke has occurred and concluded or ceased relatively recently. Thus, a family member, a friend, a patient caregiver, a clinician, an emergency service provider, or a first responder who receives the notification may take appropriate action to provide care or treatment to the patient. In some examples, the notification is further indicative of the breathing pattern. For example, in addition to indicating the stroke event, the notification may also indicate the particular breathing pattern exhibited by the patient. In some examples, the notification may further be indicative of one or more physiological parameters of the patient, for example, a heart rate, a blood pressure, a blood oxygenation level, or some other parameter. Such additional information carried by the notification may facilitate confirmation of the stroke event, or provision of appropriate care of treatment.

In some examples, computing device 14 is configured to detect the stroke event based substantially on the breathing pattern alone, or based on a combination of the breathing pattern in combination with other physiological parameters (e.g., a heart rate, a blood pressure, or a blood oxygenation level). For example, computing device 14 may detect the stroke event based on signals received from contactless sensor 12 alone, one or more additional sensors alone, or a combination of signals from contactless sensor 12 and the additional sensor(s). The additional sensor(s) may include a wearable sensor, an implantable sensor, or a contact sensor. Using a combination of contactless sensor 12 and the second sensor(s) may promote data robustness and accuracy of breathing signal collection. In some examples, the additional sensor includes a wearable device, an external medical device, or an implantable medical device. The external or implantable medical device can be configured to only sense one or more physiological parameters of a patient without therapy delivery or can also be configured to deliver therapy (e.g., electrical stimulation therapy, drug delivery therapy, cardiac therapy, or the like) to the patient. In some examples, system 10 does not include contactless sensor 12, and only includes the additional sensor(s).

Contactless sensor 12 is any suitable sensor configured to generate a signal that changes as a function of a breathing pattern of a patient without requiring direct physical contact with the patient. In some examples, contactless sensor 12 includes a Doppler sensor. For example, the contactless sensor may be configured to transmit a first energy signal 18 toward the patient and receive a second energy signal 20 from the patient. Second energy signal 20 may be generated by a reflection, scattering, or diffusion of first energy signal 18 by the patient. One or more characteristics of second energy signal 20 may differ from those of first energy signal, for example, based on the breathing pattern exhibited by the patient. In some examples, the breathing pattern may cause a frequency component of second energy signal 20 to be Doppler shifted relative to a corresponding frequency component of first energy signal 18. Thus, contactless sensor 12 may be configured to determine, by comparing first energy signal 18 and second energy signal 18, a Doppler shift indicative of the breathing pattern (e.g., a characteristic of the breathing pattern) of the patient. For example, the characteristic may include timing, amplitude, or frequency of respiration, intervals between respiration clusters, or a classification of the breathing pattern (e.g., Cheyne-Stokes, Biot's, Kussmaul, ataxic, or centralized periodic breathing). Further, contactless sensor 12 may generate, based on the doppler shift, the sensor signal.

In some examples, system 10 may include a wearable or contact sensor that contacts the patient instead of, or in addition to, contactless sensor 12. For example, the wearable or contact sensor may be configured to generate the sensor signal indicative of one or more of the breathing pattern or a physiological parameter of the patient. In some examples, a wearable device includes the wearable or contact sensor. In some examples, the wearable or contact sensor includes a Doppler sensor configured to generate the sensor signal indicative of the breathing pattern of the patient. The wearable or contact sensor may be configured to directly contact a skin of the patient. In some examples, a layer may be present between the wearable or contact sensor and the skin of the patient, for example, a layer including one or more of a clothing, a webbing, a fabric, a nonwoven material, a mesh, a bandage, an adhesive, a gel, or a lubricant. In some examples, the wearable or contact sensor may be coupled to one or more of a strap, a buckle, a belt, a housing, or a substrate that is fastened to, attached to, adhered to, or otherwise secured to the patient.

First energy signal 18 and second energy signal 20 may include any suitable energy signal capable of exhibiting a Doppler shift. For example, first energy signal 18 and second energy signal 20 may include a radiofrequency signal or an ultrasound signal. Accordingly, the doppler sensor in contactless sensor 12 may be a radiofrequency Doppler sensor or an ultrasound Doppler sensor.

Contactless sensor 12 may include one or more components configured to generate and receive energy signals. For example, contactless sensor 12 may include one or more of a transmitter, a receiver, or a transceiver 18. Transceiver 18 may be configured to generate first energy signal 18 transmitted to the patient and sense second energy signal 20 received from the patient.

One or both of contactless sensor 12 or computing device 14 may be configured to filter a signal received from the patient or the sensor signal generated by contactless sensor 12. For example, the second energy signal 20, or a Doppler shift or signal, or some further signal, may be normalized, filtered (e.g., by lowpass, highpass, bandpass, or convolution), denoised, or amplified. In some examples, one or both of contactless sensor 12 or computing device 14 include circuitry and/or software modules configured to filter or amplify one or more signals. For example, one or both of contactless sensor 12 or computing device 14 may receive at least one time series signal. Processing circuitry of computing device 14 or another device can process the at least one time series signal by at least one pattern recognition algorithm (e.g., Fourier transform, neural network, or dimensionality reduction) to identify at least one marker correlated to a risk of stroke. In some examples, the processing circuitry can apply any suitable filtering to remove noise and frequency components not of interest to reduce complexity of post processing data.

In some examples, a monitoring device 20 includes one or both of contactless sensor 12 or computing device 14. For example, monitoring device 20 may include a housing 22 including contactless sensor 12. Contactless sensor 12 may be secured to an exterior or within an interior of housing 22. In some examples, housing 22 defines a window to pass first energy signal 18 and second energy signal 20. The window may be an acoustic window, for example, including a material that permits ultrasound signals to pass through, or a radiofrequency window, for example, including a material that permits radiofrequency signals to pass through. In other examples, housing 22 itself may include a material that permits at least a portion of first energy signal 18 or second energy signal 20 to pass.

In some examples, housing 22 further includes computing device 14. For example, computing device 14 may be secured to the exterior or within the interior of housing 22. In some examples, housing 22 secures both contactless sensor 12 and computing device 14 within the interior of housing 22. In other examples, housing 22 secures contactless sensor 12, while a second housing secures computing device 14. The second housing may be secured to housing 22, or may be spaced from housing 22. In some examples, computing device 14 is removably securable to housing 22. For example, housing 22 may include a stand, a dock, or a charging station, and computing device 14 may be secured to housing 22 for charging or storage, and may be removed from housing 22, for example, to be carried, transported, or worn by the patient or another person.

Regardless of whether contactless sensor 12 and computing device 14 are positioned in the same housing 22 or different housings, in some examples, one or both of sensor 12 or computing device 14 are configured to be bedside units configured to sit proximate a bed of a patient to enabling stroke event detection while the patient is sleeping.

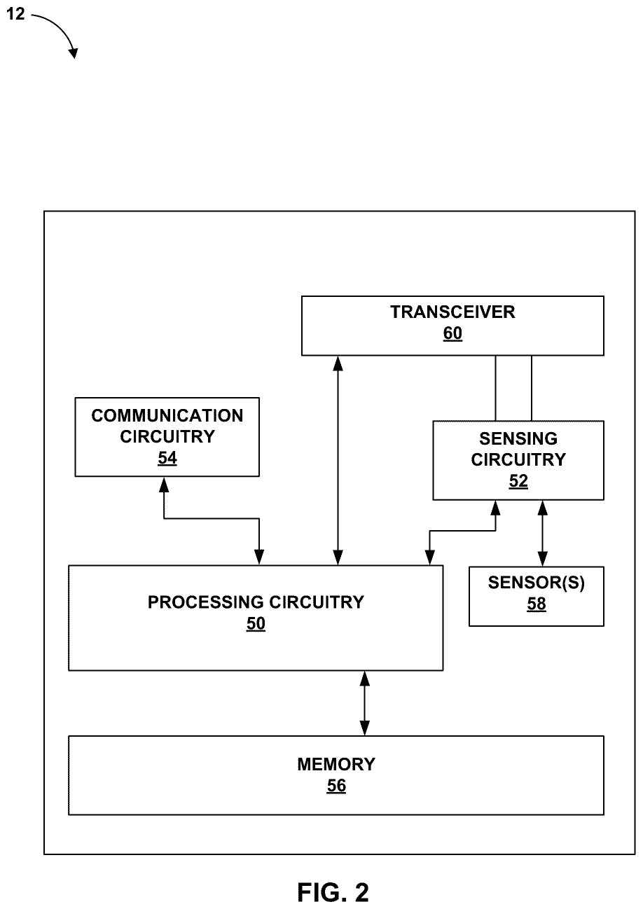

FIG. 2 is a block diagram illustrating an example configuration of the contactless sensor of FIG. 1. As shown in FIG. 2, contactless sensor 12 includes processing circuitry 50, sensing circuitry 52, communication circuitry 54, memory 56, sensors 58, and transceiver 60. In some examples, memory 56 includes computer-readable instructions that, when executed by processing circuitry 50, cause contactless sensor 12 and processing circuitry 50 to perform various functions attributed herein to contactless sensor 12 and processing circuitry 50. Memory 56 may include any volatile, non-volatile, magnetic, optical, or electrical media, such as a random-access memory (RAM), read-only memory (ROM), non-volatile RAM (NVRAM), electrically-erasable programmable ROM (EEPROM), flash memory, or any other digital media.

Processing circuitry 50 may include fixed function circuitry and/or programmable processing circuitry. Processing circuitry 50 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 50 may include multiple components, such as any combination of one or more microprocessors, one or more controllers, one or more DSPs, one or more ASICs, or one or more FPGAs, as well as other discrete or integrated logic circuitry. The functions attributed to processing circuitry 50 herein may be embodied as software, firmware, hardware, or any combination thereof.

Processing circuitry 50 is configured to control transceiver 60 to generate and transmit first energy signal 18. Sensing circuitry 52 may be coupled to transceiver 60 and controlled by processing circuitry 50. Sensing circuitry 52 is configured to monitor signal from transceiver 60 in order to determine second energy signal 20 and produce the sensor signal for the patient. In some examples, sensing circuitry 2 is coupled to one or more sensors 58 configured to sense physiological parameters of the patient, for example, heart rate, blood pressure, blood oxygenation, or other physiological parameters. The one or more sensors 58 can include respective sensing circuitry. Sensing circuitry 52 may digitize signals received from transceiver 60 and communicate the signals to processing circuitry 50 or store the digitized signals in memory 56. Thus, processing circuitry 50 may determine second energy signal 20 based on signals received through transceiver 60 and processed by sensing circuitry 52.

In some examples, one or more sensors 58 include other sensors as one or more accelerometers, microphones, optical sensors, temperature sensors, and/or pressure sensors. In some examples, sensing circuitry 52 may include one or more filters and amplifiers for filtering and amplifying signals received from one or more of transceiver 60 and/or other sensors 58. In some examples, sensing circuitry 52 and/or processing circuitry 50 may include a rectifier, filter and/or amplifier, a sense amplifier, comparator, and/or analog-to-digital converter. Processing circuitry 50 may determine physiological data, e.g., values of physiological parameters of the patient, based on signals from sensors 58, which may be stored in memory 56.

In some examples, processing circuitry 50 is configured to compare first energy signal 18 transmitted by transceiver 60 and second energy signal 20 received from the patient by transceiver 60 to generate a sensor signal. For example, the sensor signal may be indicative of a breathing pattern of the patient. In some examples, processing circuitry is configured to determine a Doppler shift in second energy signal 20 relative to first energy signal 18, and generate the sensor signal based on the Doppler shift. The sensor signal can be indicative of the Doppler shift in some examples. Processing circuitry 50 may be further configured to generate the sensor signal based on signals from sensors 58, such that the sensor signal may be indicative of one or more physiological parameters of the patient in addition to the breathing pattern. Instead of transceiver 60, contactless sensor 12 may include a transmitter and a receiver.

In some examples, processing circuitry 50 transmits, via communication circuitry 54, the sensor signal to at least one computing device 14 (FIG. 1). Such transmissions may occur continuously or intermittently, for example, over periods of seconds, minutes, or hours. Transmission of the signal may occur on an ad hoc basis and as quickly as possible. Communication circuitry 54 may include any suitable hardware, firmware, software, or any combination thereof for communicating with another device, such as computing device 14. The communication may be wireless, wired, or a combination thereof.

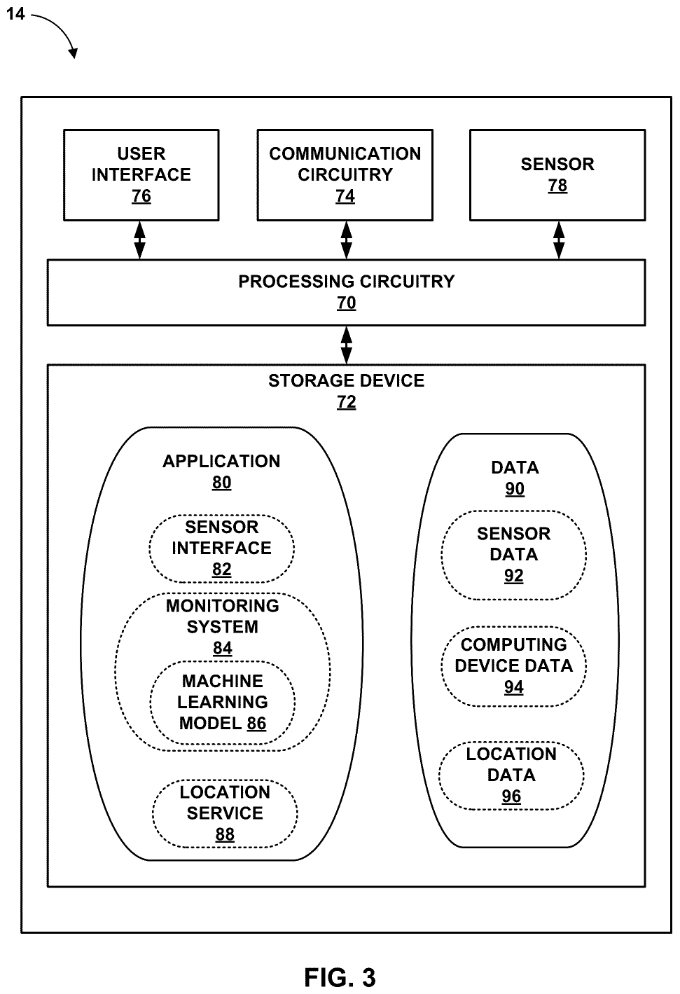

FIG. 3 is a block diagram illustrating an example configuration of computing device 14 of FIG. 1. In some examples, computing device 14 may include a smart watch, a smart phone, a tablet computer, a laptop computer, a personal digital assistant (PDA), a desktop computer, a smart speaker, a sleep monitor, a wearable computing device, a smart home appliance, or any Internet-of-Things (IoT) device. In some examples, computing device 14 and contactless sensor 12 are part of the same device.

As shown in the example of FIG. 3, computing device 14 includes processing circuitry 70, storage device 72, communication circuitry 74, and a user interface 76. While computing device 14 may be a stand-alone device as shown in FIG. 3, in other examples, computing device 14 may be any component or system that includes processing circuitry or other suitable computing environment for executing software instructions and, for example, need not necessarily include one or more elements shown in FIG. 3 (e.g., in some examples components such as storage device 72 may not be co-located or in the same housing or structure as other components).

Processing circuitry 70, in one example, is configured to implement functionality and/or process instructions for execution within at least one computing device 14. For example, processing circuitry 70 may be capable of processing instructions, including at least one application 80, stored in storage device 72. Examples of processing circuitry 70 may include, any one or more of a microprocessor, a controller, a DSP, an ASIC, a FPGA, or equivalent discrete or integrated logic circuitry.

Storage device 72 may be configured to store information within computing device 14, including at least one application 80 and data 90. Storage device 72, in some examples, is a computer-readable storage medium. In some examples, storage device 72 includes a temporary memory or a volatile memory, e.g., such as those described with respect to memory 56 (FIG. 2). Storage device 72, in one example, is used by at least one application 80 running on computing device 14 to temporarily store information during program execution. Storage device 72, in some examples, also includes one or more memories configured for long-term storage of information, e.g., including non-volatile storage elements. Examples of such non-volatile storage elements include magnetic hard discs, optical discs, floppy discs, flash memories, or forms of electrically programmable memories (EPROM) or electrically erasable and programmable (EEPROM) memories.

Computing device 14 utilizes communication circuitry 74 to communicate with other devices, such as contactless sensor 12, other computing devices 14, and computing system 30 of FIG. 1. Communication circuitry 74 may include a network interface card, such as an Ethernet card, an optical transceiver, a radio frequency transceiver, or any other type of device that can send and receive information. Other examples of such network interfaces may include 3G, 4G, 5G, and WiFi radios.

Computing device 14 may include a user interface 76. User interface 76 may be configured to provide output to a user using tactile, audio, or video stimuli and receive input from a user through tactile, audio, or video feedback. User interface 76 may include, as examples, a presence-sensitive display, a mouse, a keyboard, a voice responsive system, video camera, microphone, or any other type of device for detecting a command from a user, a sound card, a video graphics adapter card, or any other type of device for converting a signal into an appropriate form understandable to humans or machines, a speaker, a cathode ray tube (CRT) monitor, a liquid crystal display (LCD), or any other type of device that can generate intelligible output to a user. In some examples, a presence-sensitive display includes a touch-sensitive screen.

At least one application 80 executable by processing circuitry 70 of computing device 14 may include a sensor interface application 82 and a monitoring system 84 that may utilize one or more machine learning models 86. For example, monitoring system 84 may provide the sensor signal, or characteristics of the sensor signal, as inputs to machine learning models 86, and machine learning models 86 may generate an output indicative of the stroke event. For example, machine learning models 86 may utilize a binary classification, indicating a presence or an absence of a stroke event. Machine learning models 86 may be trained based on a cohort of patients. In some examples, machine learning models 86 may be trained based on the patient's historical data. In some examples, instead of, or in addition to an output indicative of the stroke event, machine learning models 86 may generate an output indicative of the particular breathing pattern of the patient (e.g., by clustering or classification). In some examples, a first machine learning model may generate a first output indicative of the particular breathing pattern, and a second machine learning model may generate a second output indicative of the stroke event. For example, an output of the first machine learning model may be provided as an input to the second learning model. In some examples, one or more physiological parameters of the patient may be provided as further inputs to the first learning model or the second learning model.

In some examples, machine learning models 86 receive at least one time series signal (e.g., from contactless sensor 12) as at least one input, and identify at least one reference breathing signature of the patient during a baseline period, for example, during a preliminary period of monitoring. Signals of interest measured during the patient's sleep cycles may be compared, for example, by machine learning models 86, with respective to the reference signals, or with respect to patient cohort data from a patient population.

Thus, processing circuitry 70 may use machine learning models 86 to determine or predict the patient's breathing pattern during a period of sleep. If processing circuitry 70 determines a deviation or anomaly in the breathing pattern, then processing circuitry 70 may process output from machine learning models 86 to determine deviation characteristics. The deviation characteristics may include one or more of magnitude of deviation from a predicted pattern, change compared to previous or historical breathing patterns or sleeping periods, a percentage of match with cohort data for characteristic breathing patterns (e.g., Cheyne-Stokes breathing), or a correlation score (e.g., on a scale of from 0 to 1, with 1 representing a high likelihood of stroke) based on the measured breathing pattern.

Execution of sensor interface 82 by processing circuitry 70 configures computing device 14 to interface with contactless sensor 12. For example, sensor interface 82 configures computing device 14 to communicate with contactless sensor 12 via communication circuitry 84. Processing circuitry 70 may receive a sensor signal or physiological data of the patient from contactless sensor 12, and store the physiological data as sensor data 92 in storage device 72. In some examples, processing circuitry 70 receives a sensor signal from contactless sensor 12 indicative of a stroke event. Sensor interface 82 also configures user interface 76 for a user to interact with contactless sensor 12 and/or sensor data 92.

Processing circuitry 70 may execute monitoring system 84 to facilitate monitoring the health of the patient, e.g., based on sensor data 92 received from contactless sensor 12 and/or computing device data 94 collected by computing device 14. Monitoring system 84 may cause processing circuity 70 and computing device 14 to perform any of the techniques described herein related to detection of a stroke event by system 10.

In some examples, processing circuitry 70 is configured to execute a location service 88 to determine the location of contactless sensor 12 or computing device 14 and, thereby, the presumed location of the patient. Processing circuitry 80 may use global position system (GPS) data, multilateration, and/or any other known techniques for locating computing devices. In some examples, computing device 14 may include the location of the patient in the notification, or generate a separate location signal, to guide a family member, a friend, a caregiver, a clinician, an emergency service provider, or a first responder to the location of the patient.

In some examples, as illustrated in FIG. 3, computing device 14 includes at least one sensor 78 configured to sense one or more physiological parameters of the patient. Processing circuitry 70 may store physiological data from sensors 78 as computing device data 94 in storage device 72. For example computing device data 104 may include one or more of activity levels, body mass, body mass index, heart rate, low, high, and/or irregular heart rate events, heart rate variability, digitized ECG, blood oxygen saturation, blood pressure (systolic and/or diastolic), respiratory rate, maximum volume of oxygen, blood glucose, peripheral perfusion, and sleep patterns.

Processing circuitry 70 may also receive user recorded health data via user interface 76 and store such data as computing device data 94. User recorded health data may include one or more of: sleep data, medical history data, quality of life data, medication taking or compliance data, allergy data, demographic data, weight, or height. Medical history data may relate to history of cardiac health, stroke, seizure, and healthcare utilization.

In some examples, processing circuitry 70 executes monitoring system 84 to perform an analysis to confirm a stroke event based on the sensor signal generated by contactless sensor 12, and delivers or withhold notifications based on the analysis. The analysis may be of sensor data 92 and/or computing device data 94. In some examples, monitoring system 84 applies the data to at least one machine learning model 86, other artificial intelligence, or other models or algorithms that do not necessarily require machine learning, such as linear regression, trend analysis, decision trees, or thresholds, to detect the stroke event based on the data.

In some examples, computing device 14 is further configured to transmit the notification to a remote system, for example, computing system 30. The remote system may include a computing device, an emergency response system, a first responder system, or a clinical system. For example, computing device 14 may be a device locally present within a vicinity of the patient, while computing system 30 may be a computing system remote from the patient (for example, a computing system that is in another room, or in another building or facility, or in a different neighborhood or any different geographic location). In some examples, computing system 30 is a secondary device belonging to the patient, a family member, a friend, a patient caregiver, a clinician, an emergency service provider, or a first responder. For example, a first device (e.g., a sleeping monitor) may include computing device 14, while a second device (e.g., a laptop computer, a smart phone, a smart speaker, a desktop computer, a cloud computing service, or a server) may include computer system 30.

FIG. 4 is a block diagram illustrating an example configuration of computing system 30 that operates in accordance with one or more techniques of the present disclosure. Computing system 30 may be configured to receive a notification from computing device 14, and forward the notification or generate a secondary notification to a recipient (e.g., a family member, a friend, a patient caregiver, a clinician, an emergency service provider, or a first responder). The secondary notification may include the notification sent by computing device 14, and may include additional information regarding the patient (e.g., the identity of the patient or the present location of the patient). Computing system 30 may be any component or system that includes processing circuitry or other suitable computing environment for executing software instructions and, for example, need not necessarily include one or more elements shown in FIG. 4. In some examples, computing system 30 may be a cloud computing system distributed across a plurality of devices.

In the example of FIG. 4, computing system 30 includes processing circuitry 102, at least one user interface (UI) device 104, communication circuitry 106, and at least one storage device 108. Computing system 30, in some examples, further includes at least one application 130 such as monitoring system 122, that are executable by computing system 30.

Processing circuitry 102, in one example, is configured to implement functionality and/or process instructions for execution within computing system 30. For example, processing circuitry 102 may be capable of processing instructions stored in storage device 108. Examples of processing circuitry 102 may include any one or more of a microprocessor, a controller, a DSP, an ASIC, a FPGA, or equivalent discrete or integrated logic circuitry.

At least one storage device 108 may be configured to store information within computing device 30 during operation. At least one storage device 108, in some examples, is a computer-readable storage medium. In some examples, at least one storage device 108 is a temporary memory, meaning that a primary purpose of at least one storage device 108 is not long-term storage. In some examples, at least one storage device 108 is a volatile memory, meaning that at least one storage device 308 does not maintain stored contents when the computer is turned off. In some examples, at least one storage device 108 is used by software or at least one application 130 running on computing system 30 to temporarily store information during program execution. At least one storage device 108 may further be configured for long-term storage of information, such as at least one application 120 and data 130. In some examples, at least one storage device 18 includes non-volatile storage elements. Examples of suitable types of memories for storage device 108 are described with respect to memory 56 (FIG. 2).

Computing system 30, in some examples, also includes communication circuitry 106 to communicate with other devices and systems, such as contactless sensor 12 or computing device 14 of FIGS. 1 to 3. Communication circuitry 106 may include a network interface card, such as an Ethernet card, an optical transceiver, a radio frequency transceiver, or any other type of device that can send and receive information. Other examples of such network interfaces may include 3G, 4G, 5G, and WiFi radios.

Computing system 30, in one example, also includes at least one user interface device 104. At least one user interface device 104, in some examples, may be configured to provide output to a user using tactile, audio, or video stimuli and receive input from a user through tactile, audio, or video feedback. At least one user interface device 104 may include, as examples, a presence-sensitive display, a mouse, a keyboard, a voice responsive system, video camera, microphone, or any other type of device for detecting a command from a user, a sound card, a video graphics adapter card, or any other type of device for converting a signal into an appropriate form understandable to humans or machines, a speaker, a cathode ray tube (CRT) monitor, a liquid crystal display (LCD), or any other type of device that can generate intelligible output to a user.

At least one application 120 may include program instructions and/or data that are executable by processing circuitry 102 of computing system 30 to cause computing system 30 to provide the functionality ascribed to it herein. Example at least one application 120 may include monitoring system 122. Other additional applications not shown may alternatively or additionally be included to provide other functionality described herein and are not depicted for the sake of simplicity.

In accordance with the techniques of the disclosure, computing system 30 receives sensor data 92 and computing device data 94 from computing device 14 via communication circuitry 106. Computing system 30 may also receive location data 96 indicating a location of the patient from computing device 14 via communication circuitry 106. Processing circuitry 102 stores such data as data 130 in storage device 108. Processing circuitry 102 may execute monitoring system 122. Monitoring system 122 may be the same as monitoring system 84 of computing device 14, e.g., computing device 14 may primarily relay messages and data to computing system 30 for performance of the techniques described herein, or may operate in combination with monitoring system 84 to facilitate any of the functionality described herein. In some examples, computing device 14 may perform certain functions described with reference to FIG. 3 (for example, determining a breathing pattern), and computing system 30 may describe further functions described with reference to FIG. 3 (for example, detecting a stroke event based on the breathing pattern, or repeating an analysis of the sensor signal to confirm the stroke event initially detected by computing device 14).

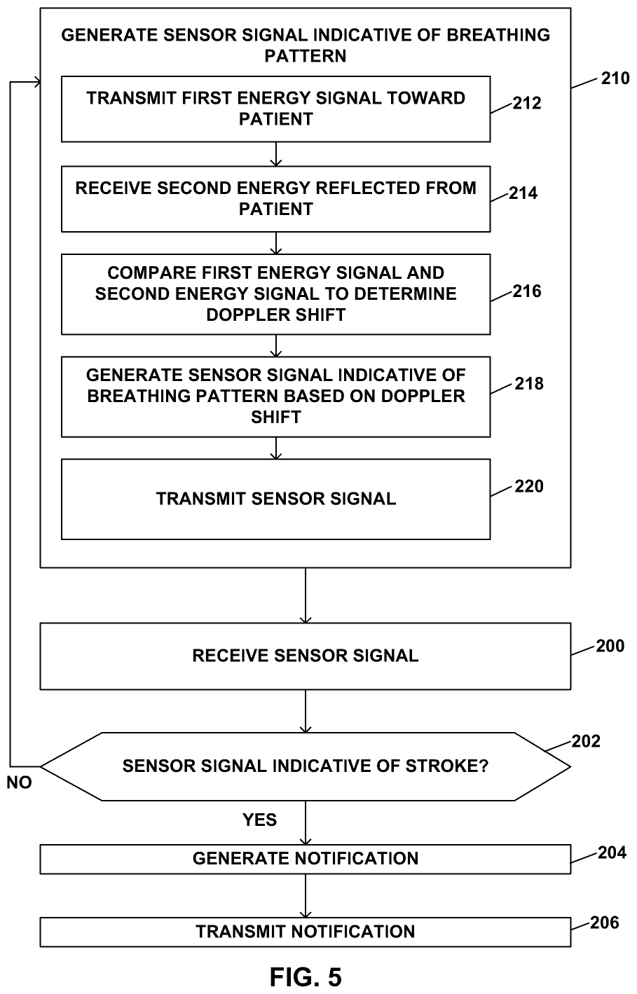

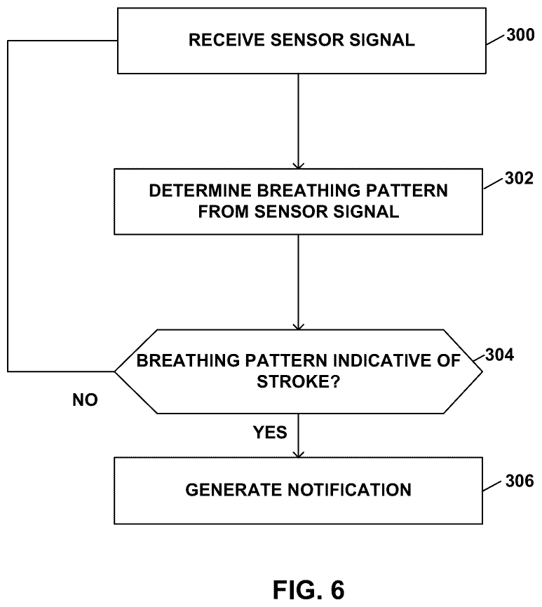

FIG. 5 is a flow diagram illustrating an example technique for detecting strokes. The example technique of FIG. 5 is described as being implemented by processing circuitry 70 of computing device 14, e.g., implementing monitoring system 84. In some examples, processing circuitry 102 of computing system 30 may implement monitoring system 122 to perform some or all of the functions of the example technique. In other examples, processing circuitry of another device alone or in combination with computing device 14 and/or computing system 30 can perform any part of the technique of FIG. 5, as well as the technique of FIG. 6.

The example technique of FIG. 5 includes receiving, by processing circuitry 70, a sensor signal indicative of a breathing pattern of a patient from contactless sensor 12 (200). The technique may further include detecting, by processing circuitry 70, based on the sensor signal, a stroke event (202). The technique may further include generating, by processing circuitry 70, in response to detecting the stroke event, a notification indicative of the stroke event (204). For example, processing circuitry 70 may analyze the sensor signal, and if the sensor signal is indicative of a stroke event (YES branch of block 202), then processing circuitry 70 generates the notification. If the sensor signal is not indicative of a stroke event (NO branch of block 202), then processing circuitry 70 may not generate the notification, and continue to monitor the patient, for example, awaiting the next instance of the sensor signal. The stroke event may include a prediction of stroke, an onset of stroke, an ongoing stroke, a ceased stroke, or a patient condition indicative of a physiological condition associated with stroke. The technique may further include, transmitting, by processing circuitry 70, the notification (e.g., an alert signal) to a remote system, for example, computing system 30, or another computing device, an emergency response system, a first responder system, or a clinical system (206).

The technique of FIG. 5 may further include generating, by contactless sensor 12, the sensor signal indicative of the breathing pattern of the patient (210). For example, contactless sensor 12 may cause transceiver 60 to transmit first energy signal 18 toward the patient (212). Contactless sensor 12 may receive second energy signal 20 generated by a reflection of first energy signal 18 by the patient, for example, via transceiver 60 (214). Contactless sensor 12 may determine, by comparing first energy signal 18 and second energy signal 20, a doppler shift indicative of the breathing pattern of the patient (216). For example, processing circuitry 70 may cause monitoring system 84 to provide first energy signal 18 and second energy signal 20 as inputs to machine learning model 86, which may generate the Doppler shift as an output. Contactless sensor 12 may further generate, based on the Doppler shift, the sensor signal (218). In some examples, processing circuitry 70 may cause monitoring system 84 to provide first energy signal 18 and second energy signal 20 as inputs to machine learning model 86, which may generate the sensor signal as an output. Contactless sensor 12 may further transmit the sensor signal to processing circuitry 70 (220), for example, via communication circuitry 74. In some examples, contactless sensor may transmit the sensor signal to another computing device (for example, computing system 30), and the another computing device may transmit the sensor signal or a remote signal indicative of the sensor signal to processing circuitry 70.

Processing circuitry 70 may detect the stroke event by directly analyzing the sensor signal, or by determining an irregular breathing pattern based on the sensor signal and detecting the stroke event based on the breathing pattern.

FIG. 6 is a flow diagram illustrating an example technique for detecting a stroke event by at least determining an irregular breathing pattern. The example technique of FIG. 5 is described as being implemented by processing circuitry 70 of computing device 14, e.g., implementing monitoring system 84. In some examples, processing circuitry 102 of computing system 30 may implement monitoring system 122 to perform some or all of the functions of the example technique or processing circuitry of another device may perform some or all of the functions of the example technique.

Part of the technique of FIG. 6 is generally similar to the technique of FIG. 5. For example, the technique of FIG. 6 also includes receiving, by processing circuitry 70, the sensor signal from contactless sensor 12 (300). The technique of FIG. 6 further includes determining, by processing circuitry 70, based on the sensor signal, the breathing pattern (302). For example, processing circuitry 70 may determine a particular breathing pattern (for example, Biot's, ataxic, Cheyne-Stokes, Kussmaul, regular, centralized periodic breathing, or any other pattern) exhibited by the patient. In some examples, processing circuitry 70 determines the particular breathing pattern by pattern matching with known patterns, or by comparing signal characteristics to predetermined thresholds. Processing circuitry 70 may further detect, based on the breathing pattern, the stroke event (304). For example, at YES branch of block 304, processing circuitry 70 may determine that the particular breathing pattern is indicative of the stroke event (for example, a centralized periodic breathing or Cheyne-Stokes breathing). At NO branch of block 304, processing circuitry 70 may determine that the particular breathing pattern (for example, a regular breathing pattern) is not indicative of a stroke event.

Processing circuitry 70 may generate a notification in response to detecting the stroke event (306). In some examples, the notification is further indicative of the breathing pattern. Thus, processing circuitry 70 may determine, based on the breathing pattern, an irregular breathing event, and based on the irregular breathing event, detect the stroke event. The stroke event may include, for example, a prediction of stroke, an onset of stroke, an ongoing stroke, a ceased stroke, or a patient condition indicative of a physiological condition associated with stroke.

The following enumerated clauses are described herein.
Clause 1: A system including: a contactless sensor configured to generate a sensor signal indicative of a breathing pattern of a patient; and processing circuitry configured to: receive the sensor signal, detect, based on the sensor signal, a stroke event, and generate, in response to detecting the stroke event, a notification indicative of the stroke event.
Clause 2: The system of clause 1, where the processing circuitry is further configured to: determine, based on the sensor signal, the breathing pattern, and detect, based on the breathing pattern, the stroke event.
Clause 3: The system of clause 2, where the notification indicates a characteristic of the breathing pattern.
Clause 4: The system of clauses 2 or 3, where the processing circuitry is further configured to: determine, based on the breathing pattern, an irregular breathing event, and detect, based on the irregular breathing event, the stroke event.
Clause 5: The system of clause 4, where the irregular breathing event includes a centralized periodic breathing event.
Clause 6: The system of any of clauses 1 to 5, where the breathing pattern is indicative of a Cheyne-Stokes breathing event.
Clause 7: The system of any of clauses 1 to 6, where the stroke event includes a prediction of stroke, an onset of stroke, an ongoing stroke, or a ceased stroke.
Clause 8: The system of any of clauses 1 to 7, where the contactless sensor includes a Doppler sensor.
Clause 9: The system of clause 8, where the contactless sensor is further configured to: transmit a first energy signal toward the patient, receive a second energy signal generated by a reflection of the first energy signal by the patient, determine, by comparing the first energy signal and the second energy signal, a Doppler shift indicative of the breathing pattern of the patient, and where the sensor signal is indicative of the Doppler shift.
Clause 10: The system of clauses 8 or 9, where the contactless sensor includes one or more of a transmitter, a receiver, or a transceiver.
Clause 11: The system of any of clauses 8 to 10, where the Doppler sensor includes a radiofrequency doppler sensor or an ultrasound doppler sensor.
Clause 12: The system of any of clauses 1 to 11, where the processing circuitry is further configured to transmit the notification to a remote system.
Clause 13: The system of clause 12, where the remote system includes a computing device, an emergency response system, a first responder system, or a clinical system.
Clause 14: The system of any of clauses 1 to 13, further including a computing device including the processing circuitry.
Clause 15: The system of clause 14, where the computing device includes a smart watch, a smart phone, a tablet computer, a laptop computer, a personal digital assistant (PDA), a desktop computer, a smart speaker, a sleep monitor, a wearable device, or a smart home appliance.
Clause 16: The system of any of clauses 1 to 15, where the contactless sensor is configured to transmit the sensor signal to the processing circuitry.
Clause 17: The system of any of clauses 1 to 15, where the contactless sensor is configured to transmit the sensor signal to a computing device, where the computing device is configured to transmit the sensor signal or a remote signal indicative of the sensor signal to the processing circuitry.
Clause 18: The system of any of clauses 1 to 17, further including a housing including the contactless sensor.
Clause 19: The system of clause 18, where the housing further includes the processing circuitry.
Clause 20: The system of clause 18, where the housing is a first housing, where the system further includes a second housing including the processing circuitry.
Clause 21: A method including: receiving, by processing circuitry, a sensor signal indicative of a breathing pattern of a patient from a contactless sensor; detecting, by the processing circuitry, based on the sensor signal, a stroke event; and generating, by the processing circuitry, in response to detecting the stroke event, a notification indicative of the stroke event.
Clause 22: The method of clause 21 further including generating, by the contactless sensor, the sensor signal.
Clause 23: The method of clauses 21 or 22 further including, by the processing circuitry: determining, based on the sensor signal, the breathing pattern; and detecting, based on the breathing pattern, the stroke event.
Clause 24: The method of clause 23, where the notification indicates a characteristic of the breathing pattern.
Clause 25: The method of clauses 23 or 24, further including, by the processing circuitry: determining, based on the breathing pattern, an irregular breathing event; and detecting, based on the irregular breathing event, the stroke event.
Clause 26: The method of clause 25, where the irregular breathing event includes a centralized periodic breathing event.
Clause 27: The method of any of clauses 21 to 26, where the breathing pattern is indicative of a Cheyne-Stokes breathing event.
Clause 28: The method of any of clauses 21 to 27, where the stroke event includes a prediction of stroke, an onset of stroke, an ongoing stroke, or a ceased stroke.
Clause 29: The method of any of clauses 21 to 28, further including, by the contactless sensor: transmitting a first energy signal toward the patient; receiving a second energy signal generated by a reflection of the first energy signal by the patient; and determining, by comparing the first energy signal and the second energy signal, a Doppler shift indicative of the breathing pattern of the patient; where the sensor signal is indicative of the Doppler shift.
Clause 30: The method of clause 29, where the first energy signal and the second energy signal each include a radiofrequency signal or an ultrasound signal.
Clause 31: The method of any of clauses 21 to 30, further including, transmitting, by the processing circuitry, the notification to a remote system.
Clause 32: The method of clause 31, where the remote system includes a computing device, an emergency response system, a first responder system, or a clinical system.
Clause 33: The method of any of clauses 21 to 32, further including, transmitting, by the contactless sensor, the sensor signal to the processing circuitry.
Clause 34: The method of any of clauses 21 to 33, further including: transmitting, by the contactless sensor, the sensor signal to a computing device; and transmitting, by the computing device, the sensor signal or a remote signal indicative of the sensor signal to the processing circuitry.
Clause 35: A non-transitory computer readable storage medium including program instructions configured to cause processing circuitry to: receive a sensor signal indicative of a breathing pattern of a patient from a contactless sensor; detect, based on the sensor signal, a stroke event; and generate, in response to detecting the stroke event, a notification indicative of the stroke event.
Clause 36: A sleep monitoring device including: a Doppler sensor configured to generate a sensor signal indicative of a breathing pattern of a patient; processing circuitry configured to: receive the sensor signal; detect, based on the sensor signal, a stroke event; and generate, in response to detecting the stroke event, a notification indicative of the stroke event; and a housing securing the Doppler sensor and the processing circuitry.
Clause 37: The sleep monitoring device of clause 36, further including an output device configured to generate an output in response to the notification.

The above detailed descriptions of examples of the technology are not intended to be exhaustive or to limit the technology to the precise form disclosed above. Although specific examples of the technology are described above for illustrative purposes, various equivalent modifications are possible within the scope of the technology, as those skilled in the relevant art will recognize. For example, while steps are presented in a given order, alternative examples may perform steps in a different order. The various examples described herein may also be combined to provide further examples. All references cited herein are incorporated by reference as if fully set forth herein.

From the foregoing, it will be appreciated that specific examples of the present disclosure have been described herein for purposes of illustration, but that various modifications may be made without deviating from the present disclosure.

Certain aspects of the present disclosure described in the context of particular examples may be combined or eliminated in other examples. Further, while advantages associated with certain examples have been described in the context of those examples, other examples may also exhibit such advantages, and not all examples need necessarily exhibit such advantages to fall within the scope of the present disclosure. Accordingly, the present disclosure and associated technology can encompass other examples not expressly shown or described herein.

Moreover, unless the word “or” is expressly limited to mean only a single term exclusive from the other items in reference to a list of two or more items, then the use of “or” in such a list is to be interpreted as including (a) any single item in list, (b) all of the items in the list, or (c) any combination of the items in the list. Additionally, the term “comprising” is used throughout to mean including at least the recited feature(s) such that any greater number of the same feature and/or additional types of other features are not precluded.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module, unit, or circuit for purposes of clarity, it should be understood that the techniques of this disclosure may be performed by a combination of units, modules, or circuitry associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” or “processing circuitry” as used herein may refer to any of the foregoing structure or any other physical structure suitable for implementation of the described techniques. Also, the techniques could be fully implemented in one or more circuits or logic elements.

Various examples have been described. These and other examples are within the scope of the following claims.

What is claimed is:

1. A system comprising:
a contactless sensor configured to generate a sensor signal indicative of a breathing pattern of a patient; and
processing circuitry configured to:
receive the sensor signal,
detect, based on the sensor signal, a stroke event, and
generate, in response to detecting the stroke event, a notification indicative of the stroke event.

2. The system of claim 1, wherein the processing circuitry is further configured to:
determine, based on the sensor signal, the breathing pattern, and
detect, based on the breathing pattern, the stroke event.

3. The system of claim 2, wherein the processing circuitry is further configured to:
determine, based on the breathing pattern, an irregular breathing event, and
detect, based on the irregular breathing event, the stroke event.

4. The system of claim 1, wherein the breathing pattern is indicative of a Cheyne-Stokes breathing event.

5. The system of claim 1, wherein the stroke event comprises a prediction of stroke, an onset of stroke, an ongoing stroke, or a ceased stroke.

6. The system of claim 1, wherein the contactless sensor comprises a Doppler sensor.

7. The system of claim 6, wherein the contactless sensor is further configured to:
transmit a first energy signal toward the patient,
receive a second energy signal generated by a reflection of the first energy signal by the patient,
determine, by comparing the first energy signal and the second energy signal, a Doppler shift indicative of the breathing pattern of the patient, and
wherein the sensor signal is indicative of the Doppler shift.

8. The system of claim 1, wherein the processing circuitry is further configured to transmit the notification to a remote system.

9. The system of claim 1, further comprising a housing comprising the contactless sensor.

10. A method comprising:
receiving, by processing circuitry, a sensor signal indicative of a breathing pattern of a patient from a contactless sensor;
detecting, by the processing circuitry, based on the sensor signal, a stroke event; and
generating, by the processing circuitry, in response to detecting the stroke event, a notification indicative of the stroke event.

11. The method of claim 10 further comprising generating, by the contactless sensor, the sensor signal.

12. The method of claim 10, further comprising, by the processing circuitry:
determining, based on the sensor signal, the breathing pattern; and
detecting, based on the breathing pattern, the stroke event.

13. The method of claim 12, further comprising, by the processing circuitry:
determining, based on the breathing pattern, an irregular breathing event; and
detecting, based on the irregular breathing event, the stroke event.

14. The method of claim 12, wherein the breathing pattern is indicative of a Cheyne-Stokes breathing event.

15. The method of claim 10, wherein the stroke event comprises a prediction of stroke, an onset of stroke, an ongoing stroke, or a ceased stroke.

16. The method of claim 10, further comprising, by the contactless sensor:
transmitting a first energy signal toward the patient;
receiving a second energy signal generated by a reflection of the first energy signal by the patient; and
determining, by comparing the first energy signal and the second energy signal, a Doppler shift indicative of the breathing pattern of the patient;
wherein the sensor signal is indicative of the Doppler shift.

17. The method of claim 10, further comprising, transmitting, by the processing circuitry, the notification to a remote system.

18. The method of claim 17, wherein the remote system comprises a computing device, an emergency response system, a first responder system, or a clinical system.

19. A sleep monitoring device comprising:
a Doppler sensor configured to generate a sensor signal indicative of a breathing pattern of a patient;
processing circuitry configured to:
receive the sensor signal;
detect, based on the sensor signal, a stroke event; and
generate, in response to detecting the stroke event, a notification indicative of the stroke event; and
a housing securing the Doppler sensor and the processing circuitry.

20. The sleep monitoring device of claim 19, further comprising an output device configured to generate an output in response to the notification.
